(19) United States
(12) Patent Application Publication
(10) Pub. No.: US 2010/0298251 A1
(43) Pub. Date: Nov. 25, 2010

(54) WOUND-HEALING AGENT CONTAINING MOMORDICAE SEMEN EXTRACT

(76) Inventors: Bongcheol Kim, Gyeonggi-do (KR); Joo-Hyun Kim, Gyeonggi-do (KR); Se-Jun Yun, Seoul (KR); Gi-Uk Jang, Seoul (KR); Sungsoo Pyo, Gyeonggi-do (KR)

Correspondence Address: FROMMER LAWRENCE & HAUG, 745 FIFTH AVENUE- 10TH FL., NEW YORK, NY, 10151, US

(21) Appl. No.: 12/739,010

(22) PCT Filed: Oct. 21, 2008
(86) PCT No.: PCT/KR2008/006219

§ 371 Date: Aug. 13, 2010

(30) Foreign Application Priority Data
Oct. 22, 2007 (KR) 10-2007-0106138

(51) Int. Cl. A61K 31/704; A61P 17/02

(52) U.S. Cl. 514/33

(57) ABSTRACT

The present invention relates to Momordicae semen extract having wound-healing efficiencies. In particular, the present invention relates to a wound-healing topical transdermal agent comprising an active ingredient of Momordicae semen extract, which is capable of reducing the time required for the closure and treatment of wounds as confirmed in skin-wound induced animal model.

### TECHNICAL FIELD

The present invention relates to Momordicae semen extract having wound-healing efficiencies. The present invention also relates to a wound-healing topical transdermal agent comprising an active ingredient of Momordicae semen extract, which is capable of reducing the time required for the closure and treatment of wounds as verified from skin-wound induced animal model.

### BACKGROUND ART

Momordicae semen is a mature seed of Momordicae, a perennial vine widely distributed over Southern China. Its fruits are collected from September to November and then treated as follows: each fruit is cut into two pieces and half-dried; seeds are removed instantly or the fruits with the seeds are put into a jar until their skins and fleshes are rotten, and the seeds are separated.

The Momordicae semen treated in this way has strong anti-inflammatory activities and treating efficiencies for rheumatoid pains and muscle spasm, etc. Until recent times, the known ingredients of Momordicae semen extract are sterol, oleanolic acid, momordic acid, momordica saponin I, II.

Nowadays, widely used external applications for treating general skin wounds are bacitracin, gentamicin, kanamycin, tetracycline, oxytetracycline, meclocycline, polymyxin, nitrofurazone, sulfadiazine, fusidic acid and the like. In fact, there has been a great demand for a novel, superior wound-healing pharmaceutical agent for effective treatment of intractable wounds such as diabetic foot ulcer. Further, a novel wound-healing agent are being developed such as functional dressing agents that compounded the above external applications with wet dressing agents such as hydrogels or polyurethane foams will create a highly value-added products.

### DETAILED DESCRIPTION OF INVENTION

### Technical Problem

The inventors of the present invention have endeavored to develop pharmaceuticals for treatment or alleviation of wound using phytochemicals having little side effects or toxicities caused by topical application compared to chemical substances. Selected crude herbal extracts were applied to wounded skin mouse model for test and the result showed that Momordicae semen extract or its fraction containing Momordica saponin I remarkably reduced the time required for the closure or treatment of wounded skin.

Therefore, the present invention aims to provide a wound-healing pharmaceutical agent comprising Momordicae semen extract or Momordicae semen fraction containing Momordica saponin I.

### Technical Solution

The present invention relates to a wound-healing pharmaceutical agent characterized by containing Momordicae semen extract as an active ingredient.

The above Momordicae semen extract comprises Momordicae semen fraction containing Momordica saponin I as an active ingredient represented by the following Formula I.

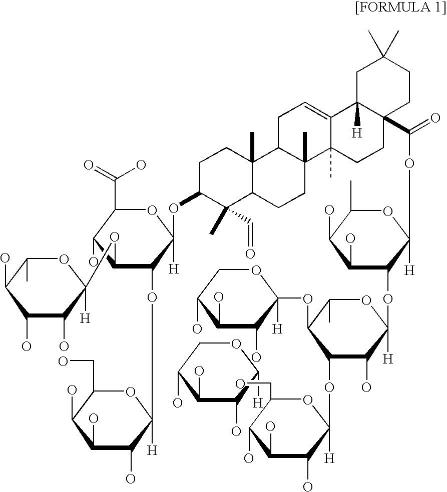

### DESCRIPTION OF DRAWINGS

FIG. 1 represents the wound healing effect shown in the wounded skin mouse model.

### BEST MODE

The present invention may be explained further herein below.

The present invention relates to a wound-healing pharmaceutical agent for topical transdermal application, comprising Momordicae semen extract or Momordicae semen fraction containing Momordica saponin I as an active ingredient, which notably reduced the time required for the closure and treatment of wounds, verified from a wounded skin animal model.

Momordicae semen extract according to present invention is obtained by extracting Momordicae semen with water or an aqueous alcohol solution with 2-10 times heavier weight than the dried Momordicae semen, and it may also be obtained by a conventional extraction method used for crude drugs. The alcohol used in the above is preferably C₁-C₆, more preferably methanol, ethanol, etc.

Momordicae semen fraction containing Momordica saponin I according to the present invention may be obtained from Momordicae semen extract, which is obtained by treating a Momordicae semen with a conventional method using a polar solvent. The Momordicae semen fraction may be effectively produced by treating the Momordicae semen extract with a conventional column chromatography using a non-ionic adsorption resin or a reverse-phase silica resin or produced by saponin sedimentation method using an organic solvent, and the organic solvent is preferably acetone, ethyl acetate, etc.

In performing the column chromatography, Amberlite XAD-16 or octadecylsilyl(ODS)-silica resin may be used with an organic solvent such as aqueous methanol solution, ethanol, acetone and the like, thereby a fraction containing highly-concentrated saponin may be selectively prepared from the Momordicae semen extract.

Momordicae semen extract is dissolved in 3-5 times (w/w) of distilled water and added with acetone 5-10 times (w/w) of greater than the water, and then saponin is selectively sedimentated, thereby finally producing a fraction containing an increased amount of momordicae saponin.

Momordicae semen extract or Momordicae semen fraction containing Momordica saponin I according to the present invention may be prepared by using a conventional method, by mixing the Momordicae semen extract or the Momordicae semen fraction containing Momordica saponin I as active ingredients, with a pharmaceutically acceptable carrier, a forming agent, a diluent, etc., in a weight ratio of 20000-20:1, and in a weight ratio of 0.01-30 wt. % relative to the amount of the whole pharmaceutical composition. In this way, the wound-healing topical transdermal agent can be prepared in the form of an ointment, a dressing agent, a patch, etc.

Further, the dosage of the extract or fraction of Momordicae semen according to the present invention varies depending on internal resorptional rate, body weight, age, sex, health condition, diet, administration time, application method, excreting rate, severity of disease, a medical expert's (or supervisor's) decision, upon a patient's request, etc.

Further, thus manufactured unit dosage preparation in this way may be applied by specialized method or may be administered at regular intervals according to a decision of a medical expert's guidance and monitoring, and upon a patient's request.

The present invention may be further described with the examples herein below but the invention is not limited to these.

### Examples

### Preparation Example 1

### Preparation of Momordicae Semen Extract

Momordicae seeds, obtained from a Chinese herb market, ground into proper size and the ground Momordicae seeds(1 kg, dry weight) were added with aqueous ethanol solution (2 L, 10%) then extracted twice for 6 hours in water bath at 80° C. The extract was filtered, concentrated under reduced pressure with a rotary evaporator at 60° C., absolutely eliminated the solvent in a vacuum oven, the resultant was dried and powdered, and then a Momordicae semen extract (40-50 g) was obtained.

### Preparation Example 2

### Preparation of Momordica Saponin I Fraction 1 by Using Non-Ionic Adsorption Resin

Column chromatography using non-ionic adsorption resin Amberlite XAD-16 was performed to the extract obtained in Preparation Example 1. The extract(30 g) dissolved in methanol(10%) was poured into adsorption resin column(1 l). Distilled water and an aqueous methanol solution (30%, v/v), respectively, were flowed through the column in the amount of 3 times the volume of the resin, and then aqueous methanol solution(70%) and 100% methanol, respectively, were flowed through the column in the amount of 3 times the volume of the resin to obtain an eluate fraction. The resultant fraction was dried, powdered, and Momordica saponin I fraction 1(10 g) was finally obtained.

### Preparation Example 3

### Preparation of Momordica Saponin I fraction 2 by Using an Organic Solvent for Precipitation

A fraction containing a high concentration of Momordica saponin I was obtained by precipitating the extract obtained in Preparation Example 1, using organic solvent such as acetone. The extract (100 g) obtained in Preparation Example 1 was dissolved in distilled water (300-500 ml), added with acetone (1800-3000 ml) and mixed together, and then a precipitate containing saponin was generated. The precipitate was separated by using a filter paper, dried, and then Momordica saponin I fraction 2 (60 g) was obtained.

### Preparation Example 4

### Preparation of Momordica Saponin I Fraction 3 by Using a Reverse-Phase Silica Resin Chromatography

Column chromatography using octadecylsilyl(ODS)-silica resin (YMC*GEL ODS-A 12 nm, S-150 m) was performed to the extract obtained in Preparation Example 3. 250 g of the resin was used relative to 10 g of a sample. After 30% (v/v) aqueous methanol solution in the amount of 2-3 times the resin was flowed, 60% (v/v) aqueous methanol solution in the amount of 2-3 times the resin was flowed to obtain an eluate fraction.

The resultant fraction was concentrated under reduced pressure, the solvent was completely dried in a vacuum oven, and then Momordica saponin I fraction 3 (3.5 g) was obtained.

### Preparation Example 5

### Isolation of Pure Momordica Saponin I

Momordica Saponin I was Purified from the Fraction Obtained in PREPARATION Example 4. High performance liquid chromatography(HLPC) using a mixed solvent of acetonitrile and water (29:71, 0.1% trifluoroacetic acid) at an elution rate of 9.5 a/min was applied and only the peak at about 45 min was collected. The resultant fraction was concentrated under reduced pressure and completely dried in a vacuum oven. The column used was YMC J′Sphere ODS-H80, and the wavelength was detected at 210 nm.

To examine the structure of the obtained material, its Mass and NMR spectrum data was compared to that of the document [Iwamoto, Okabe, Yamauchi, Tanaka, Rokutani, Hara, Mihashi, Higuchi. Studies on the constituents of Momordica cochinchinensis Spreng. I. Isolation and characterization of the seed saponins, Momordica saponin I and II. Chemical & pharmaceutical bulletin 1985, 33(2):464-478], and found out that the data was equivalent to that of Momordica saponin I, which has been reported to be present in a Momordicae semen (Momordica saponin I; 3-O-beta-D-Galactopyranosyl (1->2)-[alpha-L-rhamnopyranosyl (1->3)]-beta-D-glucuronopyranosido-28-O-beta-D-xylopyranosyl (1->3)-beta-D-glucopyranosyl (1->3)-[beta-D-xylopyranosyl (1->4)]-alpha-L-rhamnopyranosyl (1->2)-beta-D-fucopyranosyl gypsogenin).

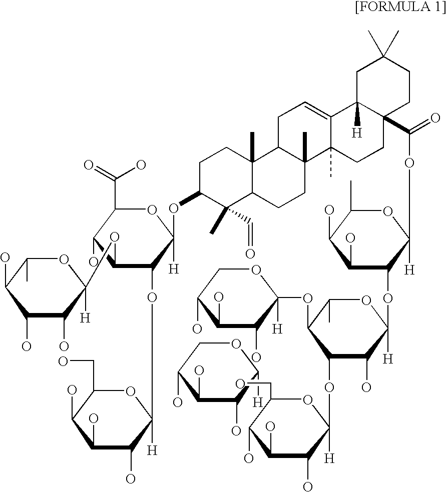

molecular weight: 1673.77

melting point: 241-244° C.

specific rotation: [α]19 D=−14.8° (C 0.7, MeOH:H₂O=1:2)

The contents of a Momordicae semen extract and Momordica saponin I fractions obtained in Preparation Example 1 to 4 are shown in Table 1.

TABLE 1

| | Content of |
|---|---|
| | Momordica |
| Classification | saponin I |
| Momordicae semen extract (Preparation Example 1) | 7-12% |
| Momordica saponin I fraction 1 (Preparation Example 2) | 21-36% |
| Momordica saponin I fraction 2 (Preparation Example 3) | 13-20% |
| Momordica saponin I fraction 3 (Preparation Example 4) | 40-50% |

### Example 1

### Wound-Healing Effect of Momordicae Semen Extract

1. Test Material and Administration

The Momordicae semen extract obtained in Preparation Example 1, the Momordica saponin I fraction 3 obtained in Preparation Example 4, and Momordica saponin I obtained in Preparation Example 5 were respectively dissolved in CMC (Carboxymethyl cellulose, 0.5%) at a concentration of 50, 10, and 5/μg/20 μl. Thus prepared test materials(20 μl) were topically applied on the wounded mouse skin, once daily for 10 consecutive days.

As a positive control drug, CGS-21680 (Tocris, USA) was prepared same as the above, and topically administered at a dosage of 10 μg/20 μl, once daily for 10 consecutive days.

2. Test Method

Twenty-five male mice of CD-1 origin having 24 g±2 g of body weight were divided into 5 groups. The mice were put into separate cages. After anesthesia with hexobarbital (90 mg/kg, IP), the furs on the shoulders and the backs of the mice were shaved. A portion of skin including a muscle layer beneath the skin and a tissue attached thereof was cut off by using a sharp punch (ID12 mm). After being wounded on the skin, the mice were administered topically with the test materials of Momordicae semen extract, Momordica saponin I fraction 3, Momordica saponin I, and CGS-21680 were, respectively, at a concentration of 50, 10, 5, and 10 μg/20 μl, respectively, once daily for 10 consecutive days.

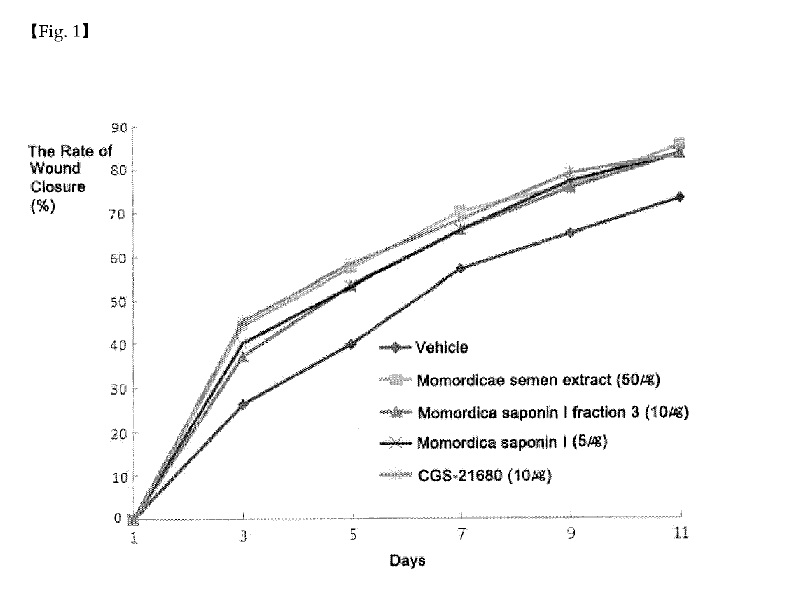

The area of wounded skin detected on a transparent plastic sheet was measured using Image-ProPlus (Media Cybernetics, Version 4.5.0.29) on day 1, 3, 5, 7, 9, and 11. Then, the wound closure rate(%) and the time required for wound closure(CT₅₀) were calculated by using a graph-prism(Graph Software USA) (Table 2 and FIG. 1). The Dunnett's test was performed after one-way analysis of variance (one-way ANOVA), in order for the comparison between the treated group and the vehicle group at each measuring point. The difference was of statistical significance at P<0.05.

As represented by Table 2 and FIG. 1, Momordicae semen extract, Momordicae semen fraction containing Momordica saponin I, and Momordica saponin I according to the present invention were discovered to have superior wound-healing effects.

TABLE 2

| | | | Wound Closure Rate (%), | | | | | | |
|---|---|---|---|---|---|---|---|---|---|
| | | | Standard Deviation(±) | | | | | | CT₅₀ |
| Content | Dosage | N | Day 1 | Day 3 | Day 5 | Day 7 | Day 9 | Day 11 | Days |
| Vehicle | (20 μl/ | 1 | 0.0 | 38.1 | 44.3 | 61.7 | 68.8 | 75.5 | 6.3 |
| (0.5% | Mouse) | 2 | 0.0 | 33.5 | 49.4 | 60.9 | 68.6 | 71.6 | 6.4 |
| CMC/PBS | x10 | 3 | 0.0 | 30.7 | 38.7 | 58.0 | 67.0 | 75.1 | 6.7 |
| pH 7.4) | | 4 | 0.0 | 16.3 | 31.4 | 50.7 | 58.4 | 72.7 | 7.6 |
| | | 5 | 0.0 | 13.0 | 35.8 | 54.1 | 64.0 | 71.0 | 7.4 |
| | | X | 0.0 | 26.3 | 39.9 | 57.1 | 65.4 | 73.2 | 6.9 |
| | | SEM | 0.0 | 4.9 | 3.2 | 2.1 | 1.9 | 0.9 | 0.3 |
| Momordica | (50 μg/ | 1 | 0.0 | 45.3 | 58.7 | 72.5 | 76.5 | 83.5 | 5.2 |
| Semen | Mouse) | 2 | 0.0 | 49.2 | 60.2 | 73.1 | 76.4 | 85.7 | 5.0 |
| Extract | x10 | 3 | 0.0 | 45.8 | 56.8 | 66.0 | 75.4 | 86.8 | 5.3 |
| (Preparation | | 4 | 0.0 | 39.1 | 51.6 | 68.7 | 76.3 | 86.6 | 5.5 |
| Example 1) | | 5 | 0.0 | 41.0 | 58.5 | 71.0 | 76.0 | 83.7 | 5.3 |
| | | X | 0.0 | *44.1 | *57.2 | *70.3 | *76.1 | *85.3 | *5.3 |
| | | SEM | 0.0 | 1.8 | 1.5 | 1.3 | 0.2 | 0.7 | 0.1 |
| Momordica | (10 μg/ | 1 | 0.0 | 30.3 | 49.5 | 69.5 | 73.8 | 78.9 | 6.0 |
| Saponin I | Mouse) | 2 | 0.0 | 37.9 | 53.6 | 68.6 | 80.9 | 89.0 | 5.4 |
| Fraction 3 | x10 | 3 | 0.0 | 36.8 | 57.3 | 71.1 | 79.1 | 84.5 | 5.4 |
| (Preparation | | 4 | 0.0 | 42.2 | 53.8 | 59.4 | 69.2 | 81.1 | 5.9 |
| Example 4) | | 5 | 0.0 | 38.8 | 52.1 | 62.2 | 74.5 | 83.2 | 5.8 |
| | | X | 0.0 | 37.2 | *53.3 | *66.2 | *75.5 | *83.3 | *5.7 |
| | | SEM | 0.0 | 1.9 | 1.3 | 2.3 | 2.1 | 1.7 | 0.1 |
| Momordica | (5 μg/ | 1 | 0.0 | 32.9 | 54.4 | 69.9 | 80.8 | 85.0 | 5.5 |
| Saponin I | Mouse) | 2 | 0.0 | 45.4 | 54.8 | 71.5 | 80.4 | 88.3 | 5.2 |
| (Preparation | x10 | 3 | 0.0 | 31.2 | 48.8 | 58.8 | 69.4 | 78.1 | 6.3 |
| Example 5) | | 4 | 0.0 | 45.6 | 51.4 | 62.9 | 75.6 | 84.6 | 5.6 |
| | | 5 | 0.0 | 45.4 | 55.5 | 67.5 | 80.5 | 81.1 | 5.3 |
| | | X | 0.0 | *40.1 | *53.0 | *66.1 | *77.3 | *83.4 | *5.6 |
| | | SEM | 0.0 | 3.3 | 1.3 | 2.3 | 2.2 | 1.8 | 0.2 |
| CGS-21680 | (10 μg/ | 1 | 0.0 | 46.7 | 62.5 | 71.2 | 79.9 | 85.6 | 5.0 |
| | Mouse) | 2 | 0.0 | 36.0 | 59.3 | 66.5 | 74.7 | 84.6 | 5.6 |
| | x10 | 3 | 0.0 | 54.4 | 55.5 | 62.7 | 79.7 | 78.3 | 5.2 |
| | | 4 | 0.0 | 46.7 | 59.6 | 71.8 | 81.6 | 82.9 | 5.1 |
| | | 5 | 0.0 | 41.6 | 55.8 | 70.3 | 79.2 | 84.3 | 5.3 |
| | | X | 0.0 | *45.1 | *58.5 | *68.5 | *79.0 | *83.1 | *5.2 |
| | | SEM | 0.0 | 3.1 | 1.3 | 1.7 | 1.2 | 1.3 | 0.1 |

(*is at P < 0.05 relative to that of the vehicle group)

### Example 2

### Acute Toxicity Test with Oral Administration to Rats

A 2-week repeated dose toxicity test was performed on 6-week-old SPF(specific pathogen free) SD rats as follows.

Momordicae semen extract obtained in Preparation Example 1 and Momordica saponin I fraction 3 obtained in Preparation Example 4, respectively, were dissolved in 0.5% CMC. The preparations were orally administered to rats (5 rats/group) with daily dosage of 2,000 mg/kg and 500 mg/kg, respectively, for 2 weeks.

On the 15th day, the rats were observed of their survival, clinical symptoms, and changes in body weight. Then, hematologic test and blood biochemical test were performed for the rats. The rats were then autopsied and observed with naked eyes to find any abnormalities on their organs of abdominal cavities and the thoracic cavities. The results showed that there were no noticeable clinical symptoms found and all the rats survived. Further, with respect to their body weight, hematologic and blood biochemical tests, and autopsies, no toxicity was found. Therefore, Momordicae semen extract and Momordica saponin I fraction 3 according to the present invention, were confirmed to be safe for oral administration, with the minimum lethal dose(LD₅₀) by oral administration of 2,000 mg/kg and 500 mg/kg, respectively.

### Formulation Example 1

### Preparation of a Transdermal Composition

A transdermal composition was prepared as described below using Momordicae semen extract or Momordicae semen fraction comprising Momordica saponin I of the present invention.

### [Composition 1]

Active ingredient (0.04 g), sodium polyacrylate (1.3 g), glycerine (3.6 g), aluminium hydroxide (0.04 g), methylparaben (0.2 g), water (14 g).

### [Composition 2]

Active ingredient (0.08 g), propylene glycol (1.6 g), liquid paraffin (0.8 g), isopropyl myristate (0.4 g), Gelva® 1430 (16.4 g).

### Formulation Example 2

### Preparation of an Ointment

An ointment was prepared as composition described below from a Momordicae semen extract or a Momordicae semen fraction comprising Momordica saponin I of the present invention.

### [Composition]

Active ingredient (1 g), cetyl palmitate (20 g), cetanol (40 g), stearyl alcohol (40 g), isopropyl myristate (80 g), sorbitan monostearate (20 g), polysorbate (60 g), propyl ρ-oxybenzoate (1 g), methyl ρ-oxybenzoate (1 g), an adequate amount of phosphoric acid and distilled water.

### INDUSTRIAL APPLICABILITY

The present invention discloses Momordicae semen extract, natural extract free of side effects or toxicities caused by topical application but with a superior effect in treating wounds, thus being expected to be used as a wound-healing pharmaceutical agent.

1. A method for the treatment of wound by administering a pharmaceutical agent which comprises Momordicae semen extract or Momordicae semen fraction containing Momordica saponin I as an active ingredient.

2. The method according to claim 1, wherein said Momordicae semen extract is obtained by extracting Momordicae semen with water or low grade aqueous alcohol solution.

3. The method according to claim 1, wherein said Momordicae semen fraction comprises Momordica saponin I increased in its content by means of saponin sedimentation method using an organic solvent or by means of column chromatography using a non-ionic absorption resin or a reverse-phase silica resin.

4. The method according to claim 3, wherein said Momordicae semen fraction contains 16-80 wt. % of Momordica saponin I represented by the Formula 1

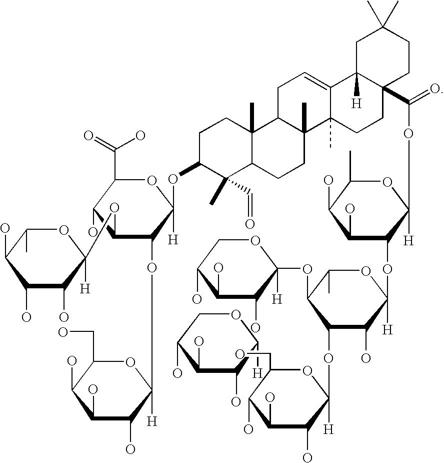

5. The method according to claim 1, wherein said agent is for a topical transdermal composition.

6. The method according to claim 5, wherein said topical transdermal composition is formulated to an ointment, a dressing or a patch.
